(19) United States
(12) Patent Application Publication
(10) Pub. No.: US 2012/0215028 A1
(43) Pub. Date: Aug. 23, 2012

(54) BIOBASED POLYOL CROSS-LINKERS FOR USE IN PREPARING POLYESTERS AND REVERSIBLE POLYURETHANES

(76) Inventors: Daniel Garbark, Blacklick, OH (US); Herman Paul Benecke, Columbus, OH (US)

(21) Appl. No.: 13/499,615

(22) PCT Filed: Sep. 30, 2010
(86) PCT No.: PCT/US10/50803

§ 371 Date: May 7, 2012

Related U.S. Application Data
(60) Provisional application No. 61/247,013, filed on Sep. 30, 2009.

(51) Int. Cl. C07C 55/02; C07C 55/22; C07C 231/02

(52) U.S. Cl. 562/590; 564/141

(57) ABSTRACT

Biobased multifunctional cross-linking polyols that can be used in preparing polyesters and in polyurethane applications, and methods of making them are described.

Cross-linkers are materials that link one polymer chain to another chain. They can be used in a wide variety of applications, but are commonly used in polymer formulations, such as epoxies, binder, adhesives, polyesters, oligomers, polyurea, and polyurethane coatings and foams. Cross-linkers can be used to control cured polymer properties, such as melting point, tensile strength, and chemical resistance. Companies are looking to increase the amount of biobased components where ever possible due to the push for “green” chemistry and cost savings.

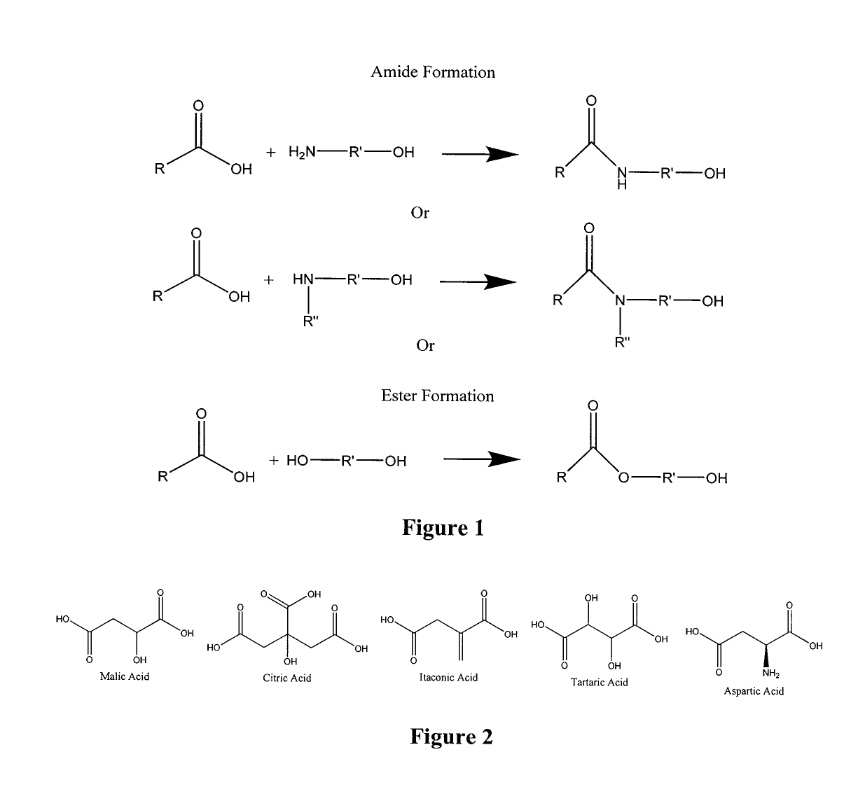

FIG. 1 is a schematic generally depicting amide and ester formation.

FIG. 2 illustrates examples of multifunctional biobased carboxylic acids showing the extra functionality such as hydroxyls, olefins, or even amines on the backbone.

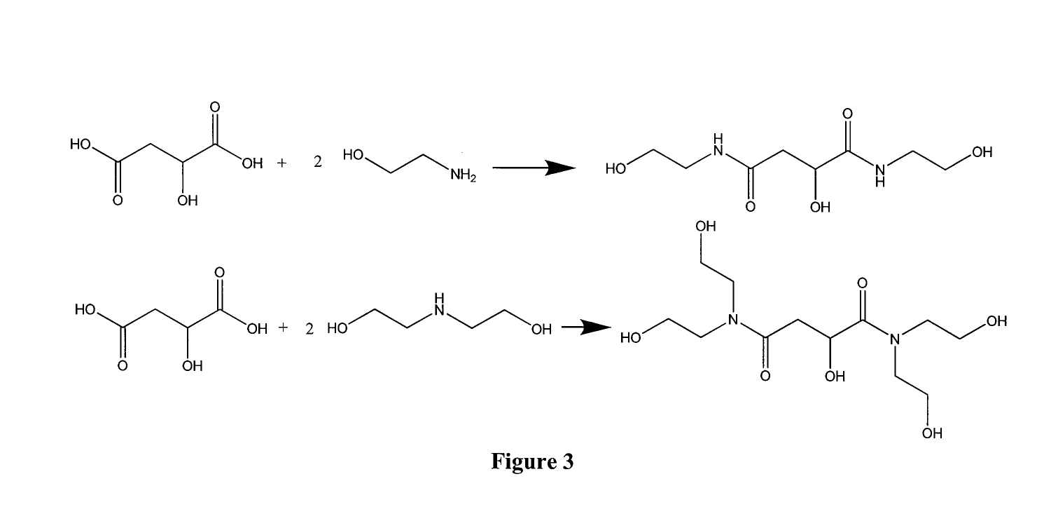

FIG. 3 is a schematic showing the reaction of a di-functional bio-based carboxylic acid (malic acid) with a mono-hydroxyl amine and a di-hydroxyl amine.

Biobased polyol cross-linkers that can be used in preparing polyesters and in polyurethane applications are described.

One aspect of the invention is a biobased multifunctional cross-linking polyol. One embodiment of the biobased multifunctional cross-linking polyol comprises a reaction product of a biobased multifunctional carboxylic acid having at least one hydroxyl group, olefinic group, or amine group on a backbone with a multifunctional alcohol or a multifunctional amine, the multifunctional alcohol having a formula HO—R′—OH, the multifunctional amine having a formula R′—NH₂ or R′NHR″, wherein R′ includes at least one hydroxyl group or amine group. R″ may or may not contain a hydroxyl or amine group.

Another aspect of the invention is a method of making a biobased multifunctional cross-linking polyol. One embodiment of the method includes reacting a biobased multifunctional carboxylic acid having at least one hydroxyl group on a backbone with a multifunctional alcohol or a multifunctional amine, the multifunctional alcohol having a formula R′—OH, the multifunctional amine having a formula R′—NH₂ or R′NHR″, wherein R′ includes at least one hydroxyl group or amine group.

By multifunctional carboxylic acids we mean that there is more than one carboxylic acid group.

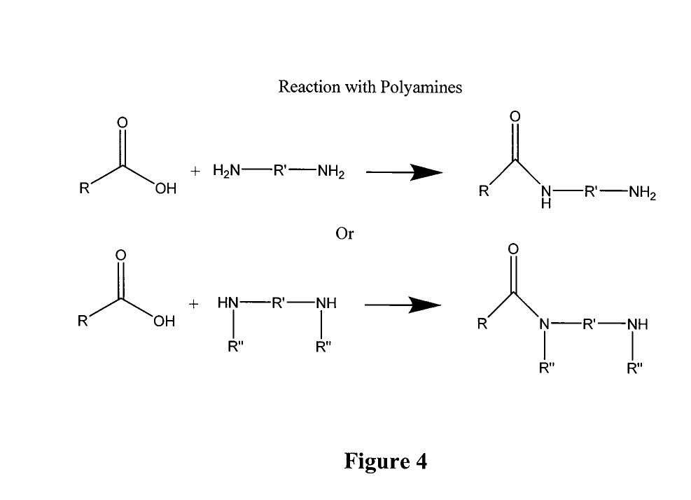

The polyol cross-linkers are derived from the amidification or esterification of biobased multifunctional carboxylic acids. The multifunctional carboxylic acids come from biobased feedstocks. FIG. 1 illustrates the general reaction of a carboxylic acid (the multifunctionality of the carboxylic acid is not shown) with an amine to form an amide or with an alcohol to form an ester. The amine can have either the formula R′—NHR″ or R′—NH₂, and the alcohol has a formula of R′—OH. The alcohol or amine includes at least one additional hydroxyl or amine group in addition to the hydroxyl or amine that forms the ester or amide. (Note that in FIGS. 1 and 4, the presence of one additional hydroxyl or amine group is shown.) These additional hydroxyl and/or amine groups are available for cross-linking reactions. R′ can be, but is not limited to, a C₁ to C₂₀ alkyl or aromatic group. R″ can be, but is not limited to, C₁ to C₂₀ alkyl, olefinic, or aromatic group and can contain, but does not have to, hydroxyl or amine functionality.

The multifunctional carboxylic acid contains one or more additional reactive groups such as hydroxyl groups, olefinic groups, and amine groups, on the backbone. The additional reactive group on the backbone does not take part in the initial ester or amide formation and is available for cross-linking reactions. Examples of multifunctional carboxylic acids with one or more additional reactive groups on the backbone are shown in FIG. 2. Suitable biobased multifunctional carboxylic acids include, but are not limited to, fumaric acid, maleic acid, itaconic acid, malic acid, citric acid, tartaric acid, aspartic acid, and glutamic acid.

The multifunctional carboxylic acids are reacted with multifunctional alcohols or amines or mixtures thereof. By multifunctional alcohols or amines, we mean that the alcohol or amine has at least one additional hydroxyl group or amine group in addition to the hydroxyl group or amine group needed to form the ester or amide linkages, respectively.

The cross-linking capacity of the polyols can be controlled by the structure of the starting materials. For example, when a bio-based di-carboxylic acid, such as malic acid, is reacted with ethanolamine (a mono-hydroxyl amine) to form a di-amide, the resulting amide polyol is tri-functional (one hydroxyl from each amide and the hydroxyl on the backbone) as shown in FIG. 3. However, if malic acid, is reacted with diethanolamine (a di-hydroxyl amine), the resulting amide polyol is pentafunctional (two hydroxyls from each amide and the hydroxyl on the backbone), as shown in FIG. 3.

If an even greater degree of cross-linking is desired, the di-acid could be reacted with a tri-hydroxyl amine, such as tris (hydroxymethyl) aminomethane, to obtain an amide polyol containing seven hydroxyl groups (three hydroxyl groups from each amide plus the one on the backbone). It should be noted that the hydroxyl groups on the amine side of the molecule seen in FIG. 3 are part of beta-hydroxyethyl amide functionality known as “primids” that have been shown to have significantly higher reactivity than “normal” primary hydroxyl groups in reactions with carboxylic acids to form ester functionality

It can be seen that if citric acid (a tri-acid with a hydroxyl group on the backbone) is reacted with diethanolamine or tris (hydroxymethyl) aminomethane, the resulting polyol would contain seven and 10 hydroxyl groups, respectively, leading to a very high degree of crosslinking while using relatively small amounts of the crosslinking agents.

If the multifunctional carboxylic acids were reacted with polyamines, then the resulting amide product would have a free amine instead of a free hydroxyl as seen in FIG. 4. This type of material would be useful in polyurea formation and epoxy curing.

The biobased multifunctional cross-linking polyols formed as described above can be further reacted with a second carboxylic acid to form a crosslinked oligomer, if desired. The second carboxylic acid could be a multifunctional carboxylic acid as described above, but it does not have to be. Nor does it need additional functionality on the backbone. Suitable carboxylic acids for reaction with the biobased multifunctional cross-linking polyols or polyamines include, but are not limited to, malic, citric, fumaric, maleic, succinic, itaconic, tartaric, furandicarboxylic acid, aspartic acid, glutamic acid, and the like.

The melting points of the multifunctional cross-linking polyols are dependent on whether the amide groups are secondary amides versus tertiary amides. The melting points can also be affected by the amount of hydroxyl content per mole. For instance, bis(N,N′-hydroxyethyl) azelamide (a secondary amide) is a low melting solid, whereas tetrakis (N,N,N′,N′-hydroxyethyl) azelamide (a tertiary amide) is a liquid at ambient temperature. These variations are presumably caused by intermolecular hydrogen bonding in the secondary amides that are not present in the tertiary amides. We also expect that the glass transition temperature (Tg) of the polyester matrices will be influenced by variations in the structure of the amide polyols used in the polyester formation wherein increasing the percent of secondary amide linkages will increase the polyester Tg.

Certain types of the biobased multifunctional cross-linking polyols can be used to make thermally reversible polyurethanes, as described in U.S. Pat. No. 6,559,263 for example, which is incorporated herein by reference. When the hydroxyl group of the polyol cross-linker is benzylic or phenolic, then the reaction with an isocyanate will be thermally reversible. For example, phenolic polyols can easily be obtained by esterifying the biobased acid with hydroquinone or amidifying with p-aminophenol.

All examples herein are merely illustrative of typical aspects of the invention and are not meant to limit the invention in any way.

### Example 1

### Amidification

Diethyl malate (24.99 g; 0.1314 mole) was added to a round bottomed flask containing 2-(methylamino)ethanol (20.74 g; 0.2761 mole), sodium methoxide (3.75 g; 0.069 mole), and methanol (50 mL). The mixture was refluxed for 1 hour with magnetic stirring. After reflux, the methanol was removed by short path distillation at 100° C. followed by use of a stream of argon to assist removal. After 2 hours, the reaction temperature was raised to 140° C. and reaction continued for 3.5 hours. The mixture was dissolved into isopropyl alcohol and purified by use of Amberlite® IR-120 resin (72 mL; 1.5 eq). The mixture was then filtered through a course fritted filter and solvent was removed by rotary evaporation followed by distillation. The resulting oil weighed 27.62 g resulting in an 84.7% yield. IR revealed amide alcohol product.

### Example 2

### Transesterification

Triethyl Citrate (40.01 g; 0.145 mole) was added to a round bottomed flask containing ethylene glycol (118.68 g; 1.91 mole) and boron trifluoride diethyl etherate (1.10 mL; 0.009 mole). The mixture was heated with magnetic stirring to 150° C. for 26 hours. The mixture was dissolved into isopropyl alcohol and purified by use of Amberlite® IRA-67 resin. The mixture was then filtered through a course fritted filter and solvent was removed by rotary evaporation followed by distillation. No ethylene glycol was needed for polyol use.

### Example 3

### Oligomerization

Polyol from example 1 [Malic Bis(2-MeEtOHAmide)] (19.46 g; 0.077 mole) was added to a round bottomed flask containing malic acid (6.90 g; 0.051 mole) and calcium hypophosphite (0.27 g; 0.01% of total weight. The mixture was heated to 140° C. for 5 hours with magnetic stirring. Removed heat and let cool. The hygroscopic solid IR spectra supported desired product by containing both amide and ester peaks at 1627 cm⁻1 and 1721 cm⁻1, respectively.

### Example 4

### Curing

A number of biobased cross-linking polyols were made. The polyols were tested for their effectiveness by mixing them with aqueous poly(acrylic acid) (PAA) with a MW (average molecular weight) of 100,000. The ratio of hydroxyl groups to carboxyl groups was 1.66. The cure time was measured on a hot plate at 180° C., and the results are shown in Table 1.

TABLE 1
Polyester Cure Testing at 180° C.

| | | Cure | Moles |
|---|---|---|---|
| Sam- | | Time | Hydroxyls |
| ple | Composition | (sec) | per Mole |
| 1 | Tri(propylene glycol) Citrate | 118 | 4 |
| 2 | Dipropylene glycol) Malate | 88 | 3 |
| 3 | Malic Bis(2-MeEtOHAmide) | 85 | 3 |
| 4 | Citric Tris(2-MeEtOHAmide) | 76 | 4 |
| 5 | Citric Tris(ethylene glycol) + | 69 | ≧2.4 |
| | ethylene glycol | | |
| 6 | FDCA Bis(diethanolamide) | 41 | 4 |
| 7 | Oligomer: FDCA Bis(2-methylethanolamide) + | 40 | 3 |
| | Malic Acid (2:1) | | |
| 8 | Oligomer: FDCA Bis(diethanolamide) + | 38 | ≧2.8 |
| | FDCA (1:0.6) | | |
| 9 | FDCA Bis(Triethanolamine ester) | 32 | 4 |
| 10 | Oligomer: Malic Bis(diethanolamide) + | 31 | ≧4 |
| | Malic Acid (1:1) | | |
| 11 | Oligomer: Malic Bis(2-methylethanolamide) + | 24 | ≧3.5 |
| | Malic Acid (1.5:1) | | |

The amide polyols proved to be more water soluble than the ester polyols prior to the reaction with PAA. The fully ester polyols were cured as suspensions in water.

The oligomers were in the range of about 50 to about 60% of the total mixture weight, which is desirable because it reduces the amount of resin (poly(acrylic acid) in this case) needed to react, increasing the total biobased content. The oligomers proved to contain the fastest cure times due to increased cross-linking.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

1. A multifunctional cross-linking polyol comprising:
a reaction product of a multifunctional carboxylic acid or carboxylic acid ester having at least one hydroxyl group, olefinic group, or amine group on a backbone with an amine having a formula R′—NH₂ or R′NHR″, wherein R′ includes at least one hydroxyl group.

2. The multifunctional cross-linking polyol of claim 1 wherein the multifunctional carboxylic acid or carboxylic acid ester comprises citric acid, malic acid, tartaric acid, itaconic acid, fumaric acid, maleic acid, aspartic acid, glutamic acid, or combinations thereof.

3. (canceled)

4. The multifunctional cross-linking polyol of claim 1 wherein the amine is a mono-hydroxyl amine, a di-hydroxyl amine, or a tri-hydroxyl amine.

5. (canceled)

6. The multifunctional cross-linking polyol of claim 1 wherein the reaction product is further reacted with a second carboxylic acid or carboxylic acid ester.

7. The multifunctional cross-linking polyol of claim 6 wherein the second carboxylic acid or carboxylic acid ester is a multifunctional carboxylic acid or carboxylic acid ester.

8. The multifunctional cross-linking polyol of claim 6 wherein the second carboxylic acid or carboxylic acid ester comprises citric acid, malic acid, fumaric acid, maleic acid, succinic acid, tartaric acid, furandicarboxylic acid, itaconic acid, aspartic acid, glutamic acid, or combinations thereof.

9. A method of making a multifunctional cross-linking polyol comprising:
reacting a multifunctional carboxylic acid or carboxylic acid ester having at least one hydroxyl group, olefinic group, or amine group on a backbone with an amine having a formula R′—NH₂ or R′NHR″, wherein R′ includes at least one hydroxyl group.

10. The method of claim 9 wherein the multifunctional carboxylic acid or carboxylic acid ester comprises citric acid, malic acid, tartaric acid, itaconic acid, aspartic acid, glutamic acid, or combinations thereof.

11. (canceled)

12. The method of claim 9 wherein the amine is a mono-hydroxyl amine, a di-hydroxyl amine, or a tri-hydroxyl amine.

13. (canceled)

14. The method of claim 9 further comprising reacting the reaction product with a second carboxylic acid or carboxylic acid ester.

15. The method of claim 14 wherein the second carboxylic acid or carboxylic acid ester is a multifunctional carboxylic acid or carboxylic acid ester.

16. The method of claim 14 wherein the second carboxylic acid or carboxylic acid ester comprises citric acid, malic acid, fumaric acid, maleic acid, succinic acid, tartaric acid, furandicarboxylic acid, itaconic acid, aspartic acid, glutamic acid, or combinations thereof.

17. The multifunctional cross-linking polyol of claim 1 wherein R′ is a C₁ to C₂₀ alkyl or aromatic group.

18. The multifunctional cross-linking polyol of claim 1 wherein R″ is a C₁ to C₂₀ alkyl, olefinic, or aromatic group.

19. The multifunctional cross-linking polyol of claim 1 wherein R″ contain a hydroxyl or amine functionality.

20. The method of claim 9 wherein R′ is a C₁ to C₂₀ alkyl or aromatic group.

21. The method of claim 9 wherein R″ is a C₁ to C₂₀ alkyl, olefinic, or aromatic group.

22. The method of claim 9 wherein R″ contain a hydroxyl or amine functionality.

23. The multifunctional cross-linking polyol of claim 1 wherein the multifunctional carboxylic acid or carboxylic acid ester is a biobased multifunctional carboxylic acid or carboxylic acid ester.

24. The method of claim 9 wherein the multifunctional carboxylic acid or carboxylic acid ester is a biobased multifunctional carboxylic acid or carboxylic acid ester.
